(19) United States
(12) Patent Application Publication
(10) Pub. No.: US 2022/0054291 A1
(43) Pub. Date: Feb. 24, 2022

(54) OFFLOADING DEVICE

(71) Applicant: Seth Huber, American Fork, UT (US)

(72) Inventor: Seth Huber, American Fork, UT (US)

(21) Appl. No.: 17/404,377

(22) Filed: Aug. 17, 2021

Related U.S. Application Data
(60) Provisional application No. 63/141,838, filed on Jan. 26, 2021.
(60) Provisional application No. 63/068,130, filed on Aug. 20, 2020.

(51) Int. Cl. A61F 5/01

(52) U.S. Cl. CPC A61F 5/0111; A61F 2005/0197

(57) ABSTRACT

An offloading device includes: a base structure configured to be disposed at least partially around a foot of a user; a securing component configured to secure around a leg of the user; and one or more offloading components coupled between the base structure and the securing component to offload weight from the foot of the user.

### RELATED APPLICATIONS

This application claims the benefit of U.S. Provisional Application No. 63/068,130, filed Aug. 20, 2020, and U.S. Provisional Application No. 63/141,838, filed Jan. 26, 2021, the contents of which are hereby incorporated by reference in their entirety.

### TECHNICAL FIELD

Embodiments of the present disclosure relate to a device, and in particular to an offloading device.

### BACKGROUND

People use their ankles and feet in many activities including moving between locations, performing tasks of employment, participating in sports activities, and so forth. The ankle and foot can have problems, such as injuries and other conditions, which cause pain, limit movement capability, and so forth.

### BRIEF DESCRIPTION OF THE DRAWINGS

The present disclosure is illustrated by way of example, and not by way of limitation, in the figures of the accompanying drawings in which like references indicate similar elements. It should be noted that different references to “an” or “one” embodiment in this disclosure are not necessarily to the same embodiment, and such references mean at least one.

FIGS. 1A-B illustrate views of an offloading device, according to certain embodiments.

FIGS. 2A-D illustrate components of offloading devices, according to certain embodiments.

FIG. 3 illustrates offloading components of an offloading device, according to certain embodiments.

FIG. 4 illustrates a securing component of an offloading device, according to certain embodiments.

FIG. 5 illustrates a base structure of an offloading device, according to certain embodiments.

FIG. 6 illustrates offloading components of an offloading device, according to certain embodiments.

FIGS. 7A-B illustrate securing components of offloading devices, according to certain embodiments.

FIGS. 8A-B illustrate securing components of offloading devices, according to certain embodiments.

FIG. 9 is a block diagram illustrating a computer system, according to certain embodiments.

FIGS. 10A-C illustrate views of an offloading device, according to certain embodiments.

### DETAILED DESCRIPTION OF EMBODIMENTS

Embodiments described herein are related to an offloading device (e.g., foot offloading device, foot-ankle offloading device, offloading orthopedic walker boot, offloading orthopedic boot, offloading foot brace, offloading foot-ankle brace, etc.).

An orthosis is an externally applied device used to modify the structural and/or functional characteristics of the neuromuscular and/or skeletal system. An orthosis may: control, guide, limit, and/or immobilize an extremity, joint, or body segment; restrict movement in a given direction; assist movement generally; reduce weight bearing forces; aid rehabilitation from fractures after the removal of a cast; and/or correct the shape and/or function of the body to provide easier movement capability or reduce pain.

A lower-limb orthosis is an external device applied to a lower-body segment to improve function by controlling motion, providing support through stabilizing gait, reducing pain through transferring load to another area, promoting better healing (e.g., for diabetic foot ulcers), correcting flexible deformities, and/or preventing progression of fixed deformities. Examples of lower limb orthoses include a walking boot (e.g., controlled ankle motion walking boot, below knee walking boot, moon boot) and a walking brace (e.g., foot ankle brace) which are orthopedic devices that can be used for treatment and stabilization of severe sprains, fractures, and tendon or ligament tears in the ankle or foot. In cases where ankle motion is to be limited, a walking boot can be used in place of a cast.

Walking boots can be used to protect and/or immobilize the foot and ankle to promote healing. Walking braces can reduce the amount of force the foot ankle complex experiences. Walking boots and walking braces can be used as a post-operative rehabilitation device, a post injury rehabilitation device, and/or treatment device for foot and/or ankle problems. Walking boots and/or walking braces may be custom fabricated.

Conventional devices have disadvantages. For example, conventional devices may take advanced fabrication skills and methods to manufacture which dramatically limits the reach of the devices. Conventional devices are designed primarily for immobilizing and protecting the foot-ankle complex while allowing most of the weight of the user to rest on the foot-ankle complex (e.g., do not take a quantifiable amount of weight off of the foot-ankle complex). Conventional devices do not quantify and/or control the amount of weight of the user that is loaded onto the foot-ankle complex. Conventional devices restrict ankle and foot movement and cause leg length imbalance. These two factors alter gait and may cause other musculoskeletal problems.

The devices, systems, and methods disclosed herein provide an offloading device (e.g., offloading walker boot, offloading foot brace, etc.).

An offloading device may include a base structure, one or more offloading components, and a securing component. The base structure is configured to contact an external surface (e.g., ground, floor, etc.). For an offloading walking boot, the base structure is disposed under the foot of the user. For an offloading brace, the base structure is disposed at least on the sides of the foot of the user.

The securing component is configured to be secured around the lower leg via one or more fasteners (e.g., hook and loop fasteners, laces, tightening buckle, ratcheting system, and/or the like).

The offloading component may include one or more springs, a motorized piston, a pneumatic device, and/or the like. The offloading component separates the securing component from the base structure. The offloading component is configured to offload at least a portion of the weight of the user from the foot-ankle complex.

The systems, devices, and methods of the present disclosure have advantages over conventional solutions. The offloading device of the present disclosure may be made without advanced fabrication skills and methods of manufacture which allows the offloading device to be available to more users. The offloading device of the present disclosure removes at least a portion of the load from the foot-ankle complex which may decrease pain, quicken recovery, and allow users to perform more activities. The offloading device of the present disclosure may be used to quantify and/or control the amount of weight (e.g., 60 pounds (lbs), 120 lbs) of the user that is to be offloaded from the foot-ankle complex. The offloading device of the present disclosure may cause less gait changes and may cause less musculoskeletal problems compared to conventional solutions.

The offloading device of the present disclosure can be manufactured using lower complexity manufacturing processes compared to conventional solutions. This allows the offloading device to be distributed widely to those whom the offloading device is of benefit.

The offloading device of the present disclosure may have adjustability of offloading which is absent in conventional solutions. The adjustability of the offloading of the offloading device allows practitioners to recommend and modify the offloading device to deliver a specified amount of offloading to speed recovery and/or mitigate the foot ankle disorder.

In some embodiments, the offloading device of the present disclosure does not use custom fitting and/or fabrication. This allows the offloading device to be distributed widely to those whom the offloading device may benefit.

In some embodiments, the offloading device of the present disclosure can be used during rehabilitation after operative procedures. This allows the user to walk with less load on the affected area. This partial offload allows for ambulation without damaging the areas in which the procedure(s) were performed. Ambulation with partial weight bearing provided by the present disclosure can improve rehabilitation time and lessen patient temporary disability.

In some embodiments, the offloading device of the present disclosure can be used to address chronic foot ankle pain. The user can selectively or exclusively use the offloading device to reduce the forces that the foot-ankle complex experiences. Reduced forces provided by the present disclosure may result in a reduction of pain experienced.

In some embodiments, the offloading device of the present disclosure can be used in the treatment of wounds, such as diabetic foot ulcers. Research shows that offloading is integral to healing foot wounds. Patient compliance to non-weight bearing orders is very low. The offloading device of the present disclosure reduces loading while allowing the user to ambulate. The offloading device improves both healing and mobility during rehabilitation.

In some embodiments, the offloading device provides offloading of the foot-ankle complex without any connection to the foot-ankle complex or with less connection to the foot-ankle complex than conventional solutions.

The offloading device may allow the foot-ankle complex to move naturally (e.g., free to move naturally). This is advantageous in rehabilitation situations and improves experience of the user which improves user compliance in use of the offloading device.

Although some embodiments of the present disclosure refer to offloading boots and offloading braces, other embodiments that include a base structure, offloading component, and securing component that are not a boot or brace may be used. Although some embodiments of the present disclosure refer to the offloading component of the offloading device including one or more springs, other embodiments may provide offloading without use of a spring.

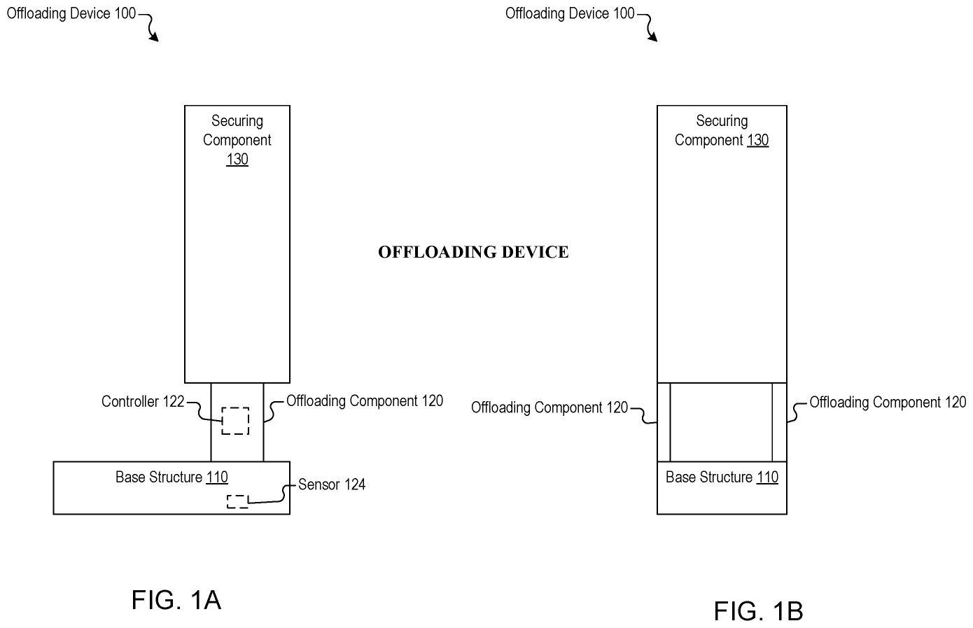

FIGS. 1A-B illustrate views of an offloading device 100, according to certain embodiments. FIG. 1A is a side view of an offloading device 100 and FIG. 1B is a front or rear view of the offloading device 100. The offloading device 100 can include a base structure 110, one or more offloading components 120, and a securing component 130.

The base structure 110 may be configured to rest on a surface, such as the ground, the floor, a surface on which the user is to be disposed (e.g., walking surface, standing surface, running surface, etc.), and/or the like. The base structure 110 provides an offset between a bottom surface of the foot and the surface under the foot (e.g., foot bed of the base structure 110, the ground, the floor, etc.) when the offloading component 120 is in an unloaded state. The bottom of the foot or component around the foot (e.g., shoe, sock, liner, etc.) may come in contact with the surface under the foot when the offloading component 120 is in a loaded state.

The base structure 110 is configured to be disposed under (e.g., the foot bed of the base structure 110 is disposed under) and/or on one or more sides of (e.g., at least partially around) the foot of the user. In some embodiments, the base structure 110 is disposed under the foot of the user (e.g., an offloading walker boot). In some embodiments, a compressible material (e.g., foam pad, neoprene) is disposed on the foot bed of the base structure 110. Responsive to the offloading component 120 being in an unloaded state (e.g., weight is not being supported by the leg), the compressible material prevents the toes of the user from contacting the foot bed of the base structure 110 (e.g., the compressible material maintains the entire foot offset from the foot bed. Responsive to the offloading component 120 being in a loaded state (e.g., weight is supported by the leg), the compressible material compresses to allow the foot to be more proximate the foot bed of the base structure 110 (e.g., the compressible material maintains the entire foot offset from the foot bed.

In some embodiments, the base structure 110 is disposed proximate the sides of the foot of the user without being disposed under the foot of the user (e.g., an offloading foot brace). In some embodiments, the base structure 110 is a U-shape that is disposed on lateral sides and behind the foot. In some embodiments, the base structure 110 has two side pieces that are configured to be disposed on either side of the foot (e.g., without being a U-shape). In some embodiments, the two side pieces of the base structure 110 are connected by a component that is configured to go over the foot.

In some embodiments, the base structure 110 is not directly connected to the securing component 130 (e.g., the securing component can be varying distances away from the base structure 110 based on compression of the offloading component 120). In some embodiments, the base structure 110 has a fixed connection (e.g., non-rotating connection, attachment plate, etc.) to the offloading component 120.

The securing component 130 may be configured to be disposed around the lower leg (e.g., shin and calf) of the user (e.g., around a portion of the lower limb between the knee to the ankle, the crus, the calf, etc.). In some embodiments, the securing component 130 includes one or more indicators (e.g., markings) indicating how much to tighten the securing component 130 for different amounts of offloading. For example, for greater offloading, the securing component 130 may secure tighter to the leg and for less offloading, the securing component 130 may secure less to the leg.

In some embodiments, to provide the offset between the bottom of the foot and the surface (e.g., floor, ground, foot bed of base structure 110) under the foot, a platform structure (e.g., rigid foam pad, wood block, etc.) is placed on the surface under the foot and the foot is placed on the platform (e.g., when weight is not being supported by the leg or the offloading device 100). The securing component 130 is then secured to the lower leg (e.g., around the shin and calf, etc.) and the platform is removed from under the foot of the user to provide the offset (e.g., gap) between the bottom of the foot and the surface under the foot. In some embodiments, the height of the offset is determined based on the amount of offloading (e.g., compression of the offloading component 120) and the amount of skin movement (e.g., amount skin stretches when secured by the securing component). In some examples, the amount of offset equals the compression of the offloading component 120 plus the distance of skin stretching. For example, for a half inch of compression of offloading component and a half inch of skin stretching, the offset (e.g., between the bottom of the foot and the surface under the foot, between the bottom of the shoe and the surface under the shoe, between the bottom of the liner and surface under the liner, the height of the platform) will be one inch.

In some embodiments, the distance of the offset between the bottom of the foot and the surface under the foot and the tightness of the securing component 130 are based on trial and error until a predetermined amount of offloading (e.g., compression of the offloading component 120 from an unloaded state to a loaded state) occurs (e.g., and there is no slipping between an inner surface of the securing component 130 and an outer surface of the leg between the unloaded state and the loaded state).

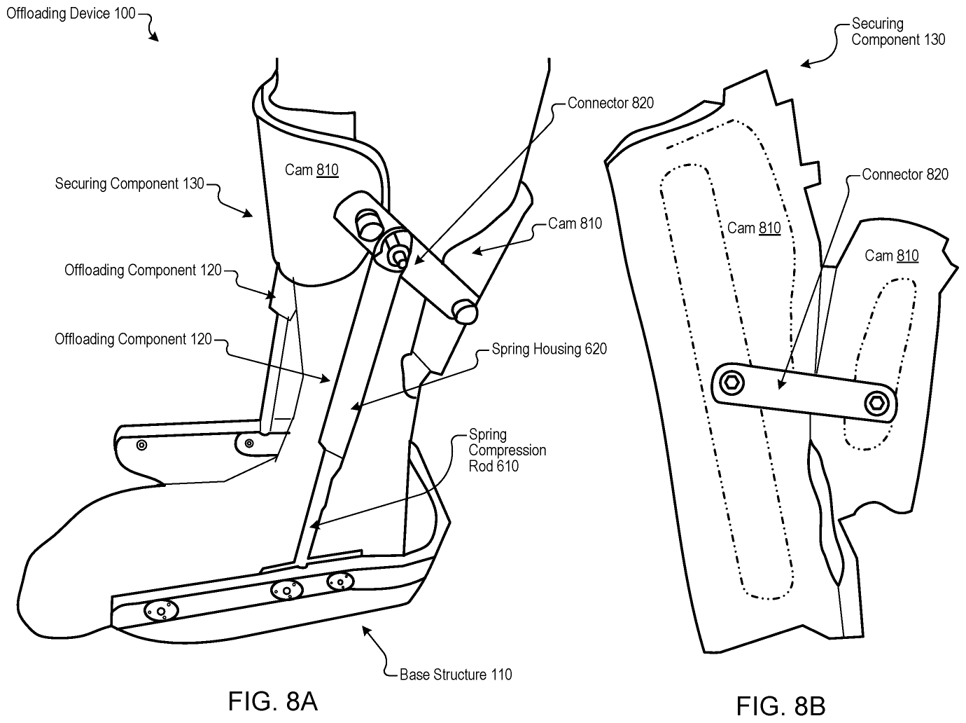

In some embodiments, the securing component 130 includes a compressible material (e.g., foam, neoprene, rubber, pneumatic bladder, silicone, etc.) and a fastener, where the compressible material is disposed between the fastener and the leg. As the fastener tightens, the compressible material compresses to avoid high pressure points and to more evenly distribute the pressure from the fastener onto the leg. The fastener can include hook and loop straps (e.g., Velcro® fastener), cams with rigid components (e.g., see FIG. 8A, different sizes and/or shapes for different legs, compressible material on inner surface of rigid components to conform to size and/or shape of leg, etc.), cams with fabric components (e.g., see FIG. 8B), laces, BOA system, tightening buckle, ratcheting system, laces that pull and are inserted into a component to secure the amount of tightening provided by the lace, and/or the like. The compressible material can include a foam or gel structure. In some embodiments, the securing component includes one or more fasteners, a compressible material, and a liner (e.g., boot liner) disposed between the fasteners and the compressible material. In some embodiments, the liner and/or compressible material is secured to the leg via multiple fasteners. In some embodiments, each of liner and/or compressible material has a single fastener.

In some embodiments, the liner is disposed around one or more of the lower leg, ankle, and/or foot. The liner may protect the lower leg, ankle, and/or foot from abrasion and/or impact.

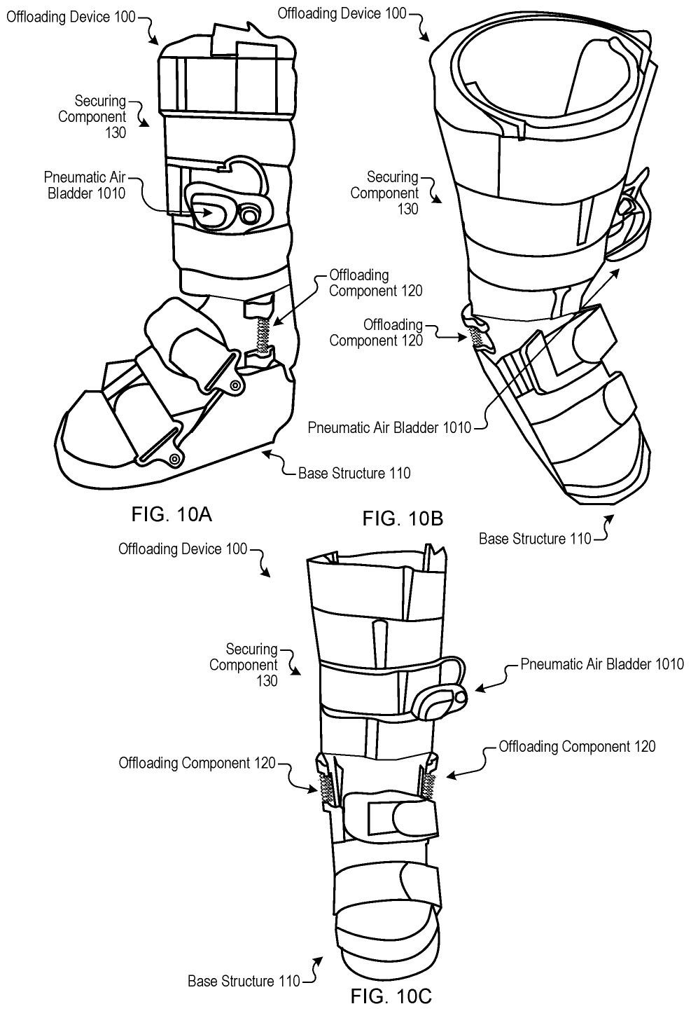

In some embodiments, the securing component 130 includes a pneumatic bladder to secure to the leg (e.g., see FIGS. 10A-C). The amount of pressure provided by the securing component 130 against the leg may be adjustable (e.g., via an input bulb and release valve) to adjust tightness of the securing component 130 around the leg. The pneumatic bladder may be filled with one or more of a fluid, gas, liquid, air, water, gel, etc.

The securing component 130 may include a supporting structure (e.g., rigid or semi-rigid structure) disposed between the offloading component 120 and the fasteners of the securing component 130 to prevent bending of the securing component 130 (e.g., of the supporting structure) during compression of the offloading component 120. The supporting structure pushes down on the offloading component 120 when in a loaded state. The supporting structure may wrap around the sides and rear of the leg. The supporting structure may include a first structure on the first side of the leg (e.g., above the first offloading component 120) and a second structure on the second side of the leg (e.g., above the second offloading component 120). The support structure may have holes to provide airflow to the leg. The supporting structure could be disposed on the front of the leg (e.g., formed to conform to the shin of the leg). The liner or compressible material may be secured to an inner surface of the supporting structure (e.g., via hooks and loops, via sewing, etc.) and the fasteners (e.g., hook and loop straps) may be secured to the outer surface of the supporting structure (e.g., via hooks and loops, via sewing, etc.).

The offloading component 120 may include one or more springs, a motorized piston, a pneumatic device, and/or the like. The securing component 130 separates the securing component 130 from the base structure 110. The securing component 130 is secured around the leg of the user with the foot elevated from the walking surface (e.g., ground, floor, inner surface of the base structure, etc.) when weight is not applied on the leg (e.g., down towards the foot-ankle complex). The offloading component 120 is configured to offload at least a portion of the weight of the user from the foot-ankle complex. In some examples, the offloading component 120 offloads about 60 to 120 lbs of weight off of the foot-ankle complex. In some examples, the offloading component 120 offloads about 30 to 180 lbs of weight off of the foot-ankle complex. In some examples, the offloading component 120 offloads about 0 to 240 lbs of weight off of the foot-ankle complex.

Responsive to the body of a user being supported by the legs of the user (e.g., standing, walking, running, etc.), the weight of the user is distributed between the two legs. Conventionally, the foot-ankle complexes of the user support the weight of the user. For example, for a user that weighs 200 lbs, 100 lbs is supported by a first foot-ankle complex and 100 lbs is supported by a second foot-ankle complex. The offloading component 120 can offset a set amount of weight from the foot-ankle complex. In the example of 100 lbs being supported by the foot-ankle complex, the offloading component 120 can be set to offload 60 lbs. When the offloading component is not supporting weight, there is a gap between a surface under the foot (e.g., ground, floor, floor bed of the base structure 110) and the bottom of the foot (e.g., shoe, liner, etc.). As the 100 lbs weight is applied to the leg, the securing component 130 pushes down on the offloading component 120 with 100 lbs (e.g., to compress the offloading component 120) and the offloading component 120 pushes back on the securing component 130 with 60 lbs. As the offloading component 120 compresses, the bottom surface of the foot comes in contact with the surface under the foot (e.g., floor, ground, foot bed of base structure). In the compressed state, 40 lbs of the 100 lbs is applied to the foot-ankle complex and 60 lbs is translated from the base structure 110 that is disposed on the ground (e.g., bypassing the foot-ankle complex), through the compressed offloading component 120, and to the securing component 130.

In some embodiments, the offloading component 120 is adjustable to provide different amounts of offloading. In some embodiments, for a first state of the foot-ankle complex, more weight (e.g., a substantial portion of the weight) is to be offloaded from the foot-ankle complex and for a second state of the foot-ankle complex, less weight is to be offloaded from the foot-ankle complex. In some embodiments, in addition to offloading weight from the foot-ankle complex, the offloading component 120 reduces the impact felt by the foot-ankle complex (e.g., softens the blow) responsive to the offloading device 100 coming in contact with the ground (e.g., the offloading component compresses and then puts a portion of the weight on the foot-ankle complex).

In some embodiments, the amount of offloading of the offloading device 100 is adjustable by adjusting offloading of the offloading component 120. In some embodiments, the offloading component 120 is adjustable by switching out the springs to different springs that have a different spring rate (e.g., different spring constant), so that with the same offset or gap, different amounts of offloading can be achieved. In some embodiments, the offloading component 120 is adjustable by adjusting the compression of the springs of the offloading component 120 in the unloaded state (e.g., adjusting separation of brackets between which the spring is disposed via tightening or loosening a screw, turning a knob, etc.) and securing the securing component 130 with the same size of gap between the foot and surface under the foot (e.g., foot bed of the base structure 110, ground, etc.). In some embodiments, the amount of offloading is adjustable by adjusting the distance between the foot and the surface under the foot (e.g., foot bed of the base structure 110, ground, etc.) to adjust the amount of compression of the offloading component 120 when weight is supported by the leg.

In some embodiments, there is an offloading component 120 on opposing sides of the leg of the user. This may provide more stability, more even offloading, more protection of the foot-ankle complex, and so forth. In some embodiments, there is an offloading component 120 at the rear of the leg. This may provide offloading for the heel of the user.

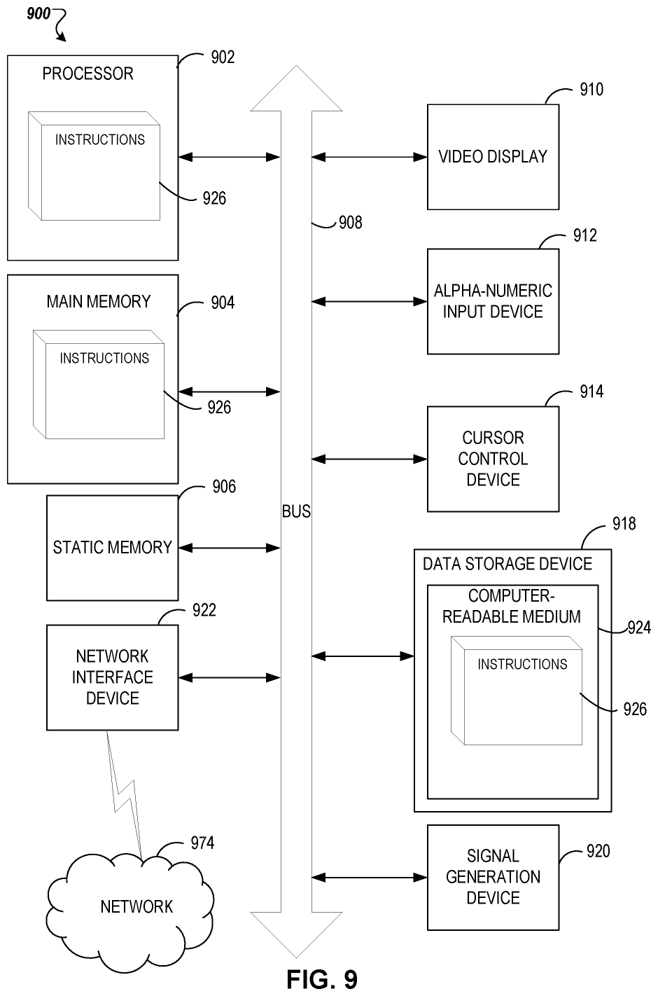

In some embodiments, offloading device 100 has a controller 122. The controller 122 may include one or more components of computer system 900 of FIG. 9. The controller 122 may receive user input (e.g., via one or more buttons, a liquid crystal display (LCD)), via a wireless component, etc.) and may cause the offloading component 120 to adjust amount of offloading based on the user input. In some embodiments, the controller 122 receives sensor data (e.g., pressure data from a pressure sensor in the base structure 110, distance data from a sensor in the base structure 110, etc.) from a sensor 124 and controls the amount of offloading based on the sensor data. In some embodiments, the controller 122 controls the amount of offloading based on user input and sensor data.

In some embodiments, the offloading component 120 includes one or more electrical components that are controlled by the controller 122. In some examples, the offloading component 120 includes a motor that adjusts spring load, a piston that adjusts spring load, an electrical actuator (e.g., instead of or in addition to a knob or a screw) that adjusts compression of the offloading component 120 when in an unloaded state, a pneumatic piston, etc.

In some embodiments, the securing component 130 includes one or more electrical components coupled to the controller 122, such as an electronic valve on a pneumatic bladder, a sensor 124 (e.g., a pressure sensor, a heart rate sensor), etc. In some embodiments, the base structure 110 includes one or more electrical components coupled to the controller 122, such as a sensor 124 (e.g., pressure sensor, a distance sensor, a heart rate sensor), etc.

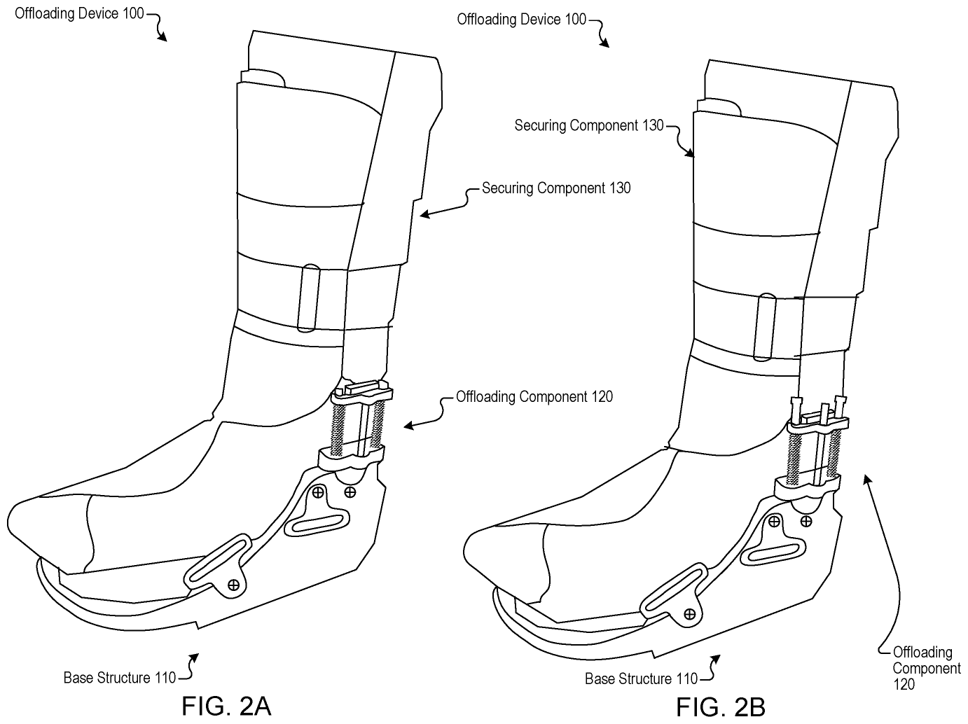

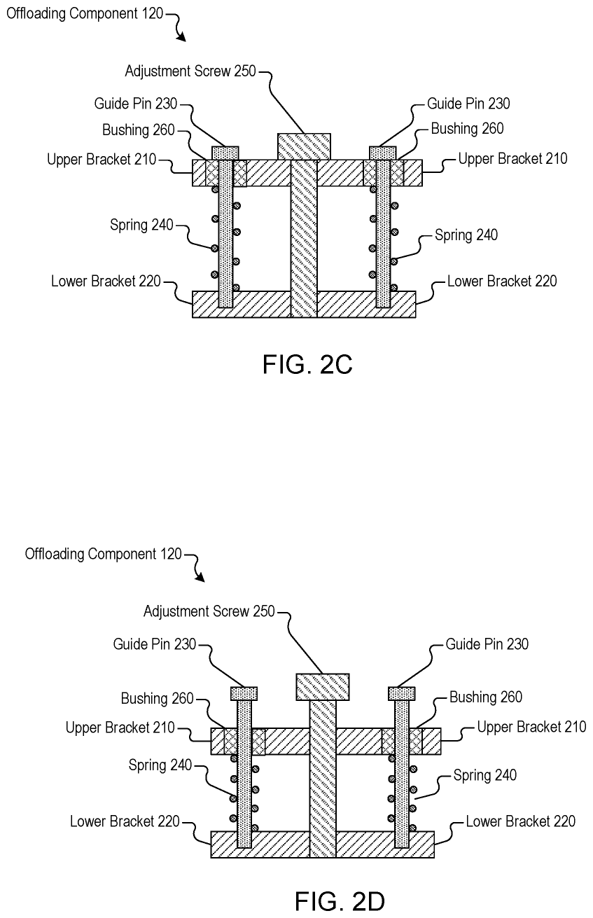

FIGS. 2A-D illustrate components of offloading devices 100 (e.g., offloading walking boot), according to certain embodiments. FIG. 2A illustrates the offloading device 100 without weight applied (e.g., without weight applied on the leg in a downward direction, offloading component 120 in an uncompressed state, in an unloaded state). FIG. 2B illustrates the offloading device 100 with weight applied (e.g., with weight applied on the leg in a downward direction, offloading component 120 in a compressed state, a loaded state). In FIG. 2B, the foot (e.g., and bottom surface of the liner) is proximate (e.g., rests on) the foot bed of the base structure 110 and the offloading component 120 is compressed (e.g., springs are compressed). FIG. 2C illustrates an offloading component 120 in an uncompressed state (e.g., unloaded state) and FIG. 2D illustrates an offloading component 120 in a compressed state (e.g., loaded state).

The offloading device 100 (e.g., offloading orthopedic walker boot) may include a base structure 110 that is a rockered base constructed of plastic with a rubber outsole and foam foot bed. The offloading device 100 may include two offloading components 120 (e.g., two spars) that protrude out of the base structure 110 on either side of the leg of the user. Each offloading component 120 (e.g., spar) has an offloading mechanism integrated into the offloading component 120. The offloading components 120 may connect to a soft fabric and foam boot liner that protects and cushions the lower leg and foot-ankle complex.

Conventional devices do not include offloading components 120 (e.g., offloading mechanisms). Conventional devices also do not include the fabric and foam liner (e.g., of the securing component 130).

In some embodiments, the offloading component 120 (e.g., offloading mechanism) includes an upper bracket 210, a lower bracket 220, guide pins 230, springs 240, an adjustment screw 250, and bushings 260. The lower brackets 220 are attached to the lower spars and the upper brackets 210 are attached to the upper spars. The lower bracket 220 has two holes (e.g., recesses, etc.) into which the guide pins 230 are attached. The lower bracket 220 also has a threaded hole that accommodates the adjustment screw 250. The upper bracket 210 has three unthreaded holes in it, where two of the holes accommodate bushings 260 and the other hole accommodates the adjustment screw 250. The springs 240 are installed by sliding the springs 240 on top of the guide pins 230 which keeps the springs 240 constrained and prevents spring buckling. Additionally, the guide pins 230 align with and slide through the bushings 260 of the upper bracket 210. The upper bracket 210 “floats” on the springs constrained in the negative z-dimension by the maximum travel of the springs 240 and in the positive z-direction by the adjustment screw 250.

In some embodiments, the offloading component 120 has a different number of guide pins 230, bushings 260, springs 240, etc. In some examples, the offloading component 120 has one guide pin 230 and spring 240 per side of the offloading device 100 (e.g., instead of two guide pins 230 and springs 240 for each bracket 210, 220).

In some embodiments, the offloading component 120 uses an elastic material (e.g. carbon fiber spar, fiberglass rod, etc.) that either deflects or extends as the offloading device 100 is loaded to achieve an offloading effect. The elastic material may include a portion of the boot spar(s) including a material such as carbon fiber that is configured to flex outwards upon loading acting as a spring and providing offloading.

In some embodiments, the offloading component 120 includes a motor that exerts a force that translates a portion of the weight user away from the foot-ankle complex.

In some embodiments, the amount of offloading of the offloading component 120 is adjusted via a screw that compresses the springs which causes an increased load to further compress the springs, this increases the offloading amount. In some embodiments, adjustment of the amount of offloading (e.g., of the offloading component 120) that the offloading device 100 provides includes one or more of: changing the springs which allows springs with different spring rates to be used (e.g., those adjusting the amount of offload provided); engaging or disengaging springs in the system; a knob that is configured to compress the springs; donning the boot with varying heights between the foot bed and the foot of the user, such as different thicknesses of platforms that could be place on the foot bed of the base structure 110 when the offloading device 100 is used; a ratcheting system that increases spring tension or compresses springs; and/or the like.

The offloading device 100 may include a boot liner that includes fabric and compressible material (e.g., foam, gripping fabric, coating, gel) that is attached together in a pattern that wraps around the foot and ankle and lower leg to just below the knee. The boot liner is configured to protect the foot, ankle, and leg and to distribute the forces that are placed on the lower leg comfortably and without slipping. The compressible material used is an impact resistant compressible material that distributes loads very well. The compressible material may be the contact surface between a leg of the user and the boot liner. The compressible material mitigates slipping and comfortably distributes the offloading load by conforming to the leg. The fabric is attached to the compressible material to allow the compressible material to conform to the leg of the user. The fabric has loop material on the outside which is how the boot liner attaches to the boot spars which have hook fabric affixed to them. The boot liner lower portion is lightly secured together by a strip of hook and loop. The upper portion of the boot liner that is around the lower leg is secured by hook and loop cinch straps that are attached to be configured to pull the liner together (e.g., like the lacing of a shoe, rather than just going around the liner). This helps to create and maintain the proper amount of pressure on the leg to prevent significant slipping of the liner up the leg when loaded.

In some embodiments, the boot liner (e.g., securing component 130) that enables an offloading effect includes one or more of: a custom upper constructed out of fiberglass or carbon fiber (e.g., similar to sockets created to support prosthetic limbs); an upper portion including materials that may be formed to the leg of the user (e.g., heat softening plastics); an upper portion including leather and compressible material (e.g., foam, gripping fabric, coating, gel) that conforms to the leg of the user; fabric and compressible material (e.g., foam, gripping fabric, coating, gel, silicone) upper portion integrated with one or more inflatable air bladders; and/or the like.

In some embodiments, the securing component 130 includes one or more of: hook and loop (e.g., Velcro®) cinching straps; spin laces (e.g., BOA fit system); laces; buckles; and/or the like. To maintain and adjust the tightness of the liner, the offloading device 100 may include one or more of: hook and loop fasteners; laces pulled through a ratcheting device; laces pulled tight and then prevented from loosening by a cam-ing mechanism or locking cleat; and/or the like.

In some embodiments, to use the offloading device 100, the liner (e.g., boot liner) is securely attached to the leg of the user and then the liner is attached to the boot spars (e.g., offloading component 120 so that the foot is suspended above the foot bed of the offloading device 100. This distance between the foot bed and the foot is based on the amount of offloading that is desired, the spring travel distance, and/or a platform of specific height. When the user walks or stands and thereby loads the offloading device 100, the springs compress and transfer load to the lower leg. As more load is added, the foot of the user eventually contacts the foot bed of the offloading device 100. The amount of offloading then stops increasing and stays at a semi-constant amount (e.g., the amount of offloading may change in different phases of gait).

Referring to FIG. 2A, the offloading device 100 (e.g., offloading boot) includes a liner (e.g., boot liner) that holds the foot and ankle above the foot bed. In some embodiments, a structure (e.g., foam, such as a soft foam that compresses easily) is placed on the foot bed to provide offsetting of the foot from the foot bed when securing the offloading device 100 to the user. When weight is put onto the limb the offloading component 120 (e.g., springs on the boot spars) compress which provides an increasing amount of offloading until the foot of the user contacts the foot bed of the offloading device 100.

In some embodiments, the base structure 110 includes one or more materials that are stiff and strong enough to enables transfer of load from the ground to the offloading component 120 and then from the offloading component 120 to the upper portion (e.g., securing component 130, brace upper) of the offloading device 100.

In some embodiments, the base structure 110 has a rocker geometry at the bottom of the base structure that may mimic the roll of natural gait.

In some embodiments, the base structure 110 is constructed of one or more of plastic, foam, aluminum, carbon fiber composite, plastic, fiberglass composite, wood, and/or metal. The bottom of base structure 110 may have material or coating to increase grip, such as a rubber coating.

The liner (e.g., boot liner) may connect to one or more portions of the offloading device 100 (e.g., the securing component 130) via hook and loop fasteners.

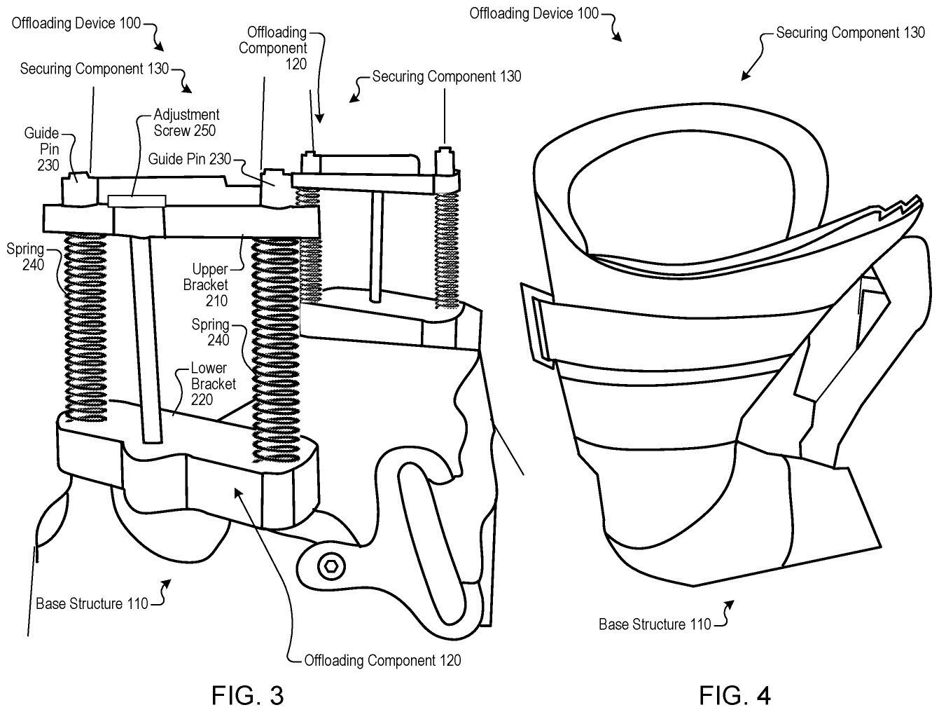

FIG. 3 illustrates offloading components 120 of an offloading device 100, according to certain embodiments. In some embodiments, the offloading component 120 includes upper bracket 210, lower bracket 220, guide pins 230, bushings, springs 240, and an adjustment screw 250.

The offloading component 120 may take the load of ambulation and transfer part of the load around the foot and ankle to the lower leg.

Responsive to the offloading device 100 (e.g., offloading boot) being loaded, the offloading component (e.g., springs 240) may compress and the foot of the user rests on the foot bed. The offloading device 100 translates a portion of the weight to the lower leg.

In some embodiments, the offloading component 120 is made from one or more of steel, aluminum, and composite bushings, plastic, fiberglass composite, carbon fiber composite, and other metals.

FIG. 4 illustrates a securing component 130 of an offloading device 100, according to certain embodiments. In some embodiments, the offloading device 100 includes a liner (e.g., boot liner) that has a foam and fabric construction.

The liner attaches to the lower leg and to the securing component 130. The liner securely and comfortably holds the lower leg of the user so that part of the load of ambulation can be transferred around the foot and ankle to the lower leg.

The liner may include a foam layer (e.g., seamless foam layer) that prevents the liner from slipping when loaded by gripping the skin of the user and conforming to the leg responsive to the liner being tightened. The foam layer may also transfer the load from the securing component 130 (e.g., tightening medium, straps) which makes the liner more comfortable and prevents harm to the user.

In some embodiments, the securing component 130 (e.g., straps) is positioned and secured to the outside surface of the liner so that the securing component 130 pulls the liner together (e.g., pulls the liner together evenly to distribute pressure similar to laces of a shoe pulling the shoe together evenly distributing pressure).

The outer surface of the liner may include a loop fabric that removably attaches to hook fabric attached to the securing component 130 (e.g., boot base spars).

The securing component 130 (e.g., straps) may provide compression that assists in securing the offloading device 100 to the leg of a user. The securing component 130 (e.g., straps) may be sewn to an upper portion of the offloading device 100 or may be independent of the upper portion of the offloading device 100. The securing component 130 may include a variable number of straps depending on the amount and location of compression to be applied. In some embodiments, this compression is provided by one or more of straps, lacing, and/or a pneumatic bladder.

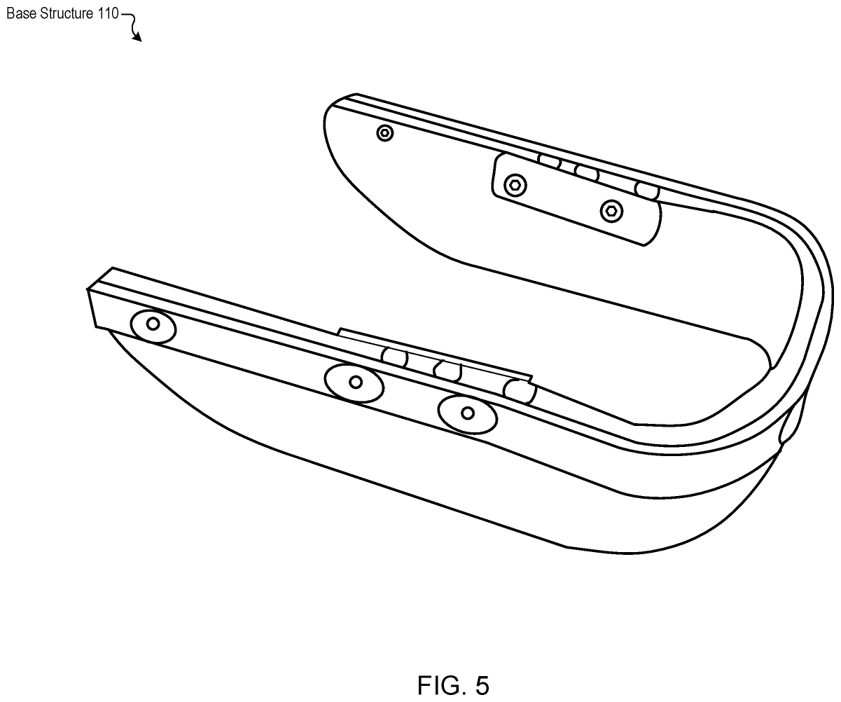

FIG. 5 illustrates a base structure 110 of an offloading device 100 (e.g., offloading boot, offloading brace), according to certain embodiments. The base structure 110 may be a rigid base that has a U-shape, where the base structure 110 is positioned around the foot (e.g., proximate the sides and rear of the foot) without being connected to the foot (e.g., without contacting the foot, without being under the foot, etc.). The offloading device 100 may include rods that transfer load from the base structure 110 to an offloading component 120 that includes springs that are in a housing. The offloading component 120 (e.g., housing that houses the springs) is connected to the lower leg of a user by a securing component 130 (e.g., tightening calf sleeve). The amount of offloading that the offloading component 120 is to provide may be adjusted (e.g., the spring housing includes a feature for adjusting amount of offloading that the spring is to provide).

When using the offloading device 100 (e.g., brace), the user positions the offloading device 100 so that the foot is suspended above the surface (e.g., ground, floor) on which the base structure 110 is disposed (e.g., the bottom of the base structure 110 is below the bottom of the shoe of the user). This distance corresponds to the amount of offloading to be provided and the spring travel distance. Responsive to walking or standing with the offloading device 100, the base structure 110 contacts the ground first and force is translated to the lower leg. As more load is added, the foot of the user contacts the ground and the amount of offload stops increasing and stays at a semi-constant amount (e.g., the amount of offloading may change in different phases of gait).

Referring to FIG. 5, the base structure 110 may be disposed around (e.g., on lateral sides of) the foot (e.g., shoe) of the user. A portion (e.g., a bottom surface) of the base structure 110 may sit below the bottom of the shoe or foot of the user when the offloading device 100 is unweighted.

The base structure 110 may include material that allows transfer of load from the ground to the upper portion (e.g., securing component 130) of the offloading device 100.

The base structure 110 does not connect with the foot or ankle in a load bearing manner. The base structure 110 is rigid enough to pass the load to the offloading component 120 (e.g., spring mechanism) without significant deflection.

A bottom surface of the base structure 110 may have a rocker geometry to mimic the roll of natural gait.

In some embodiments, the base structure 110 has a U-shape (e.g., see FIG. 5). In some embodiments, the base structure 110 includes a first runner on a first side of the foot and a second runner on a second side of the foot that are connected by a component (e.g., rigid component) that is configured to be disposed over the foot (e.g., connected by a piece that goes over the foot).

In some embodiments, the base structure 110 is constructed of one or more of acrylonitrile butadiene styrene (ABS) plastic (e.g., thermoformed ABS plastic), shaped aluminum, carbon fiber composite, plastic, fiberglass composite, wood, and/or metal. The bottom surface (e.g., bottom edge) of the base structure 110 can include a material or coating to increase grip, such as a rubber coating or rubber strip.

In some embodiments, the base structure 110 is connected to the offloading component 120 (e.g., spring mechanism) via a hole in the base structure 110 into which the spring compression rod is inserted. In some embodiments, the base structure 110 is connected to the offloading component 120 (e.g., spring compression road) via a hinge, a clamp, welding, adhesive, and/or the like.

In some embodiments, the base structure 110 includes multiple holes that allow adjustability of the attachment point of the offloading component 120 (e.g., via the spring compression rod).

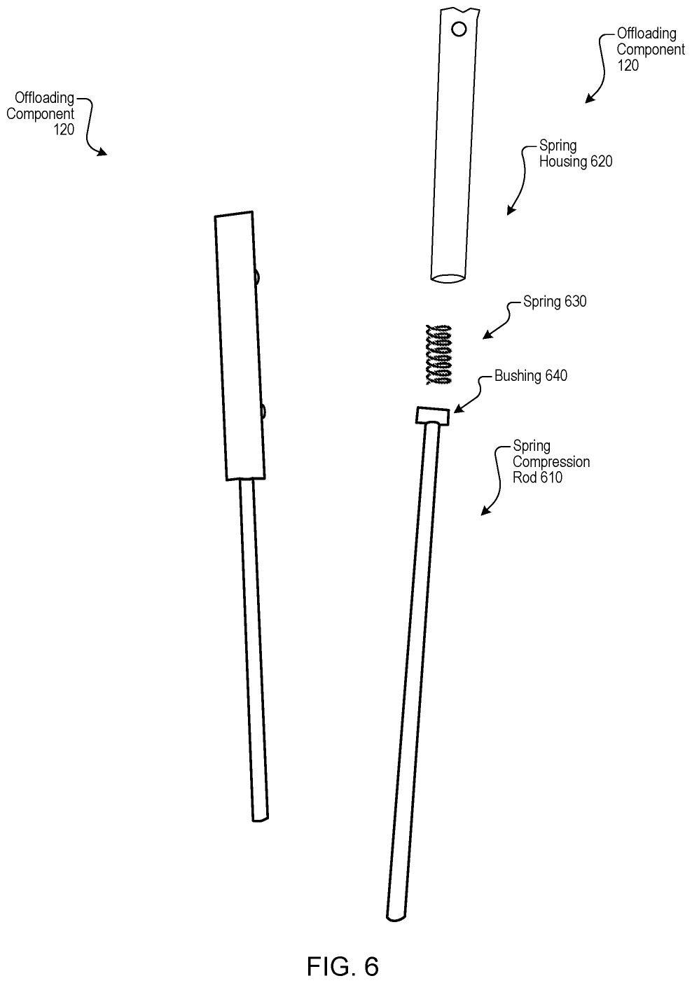

FIG. 6 illustrates offloading components 120 of an offloading device 100, according to certain embodiments. The offloading component 120 (e.g., spring offloading mechanism) may include a spring compression rod 610, spring housing 620, and spring 630 (e.g., spring 240).

The offloading component 120 takes load from the base structure 110 and transfers the load to a spring 630 or spring-like device.

Responsive to the offloading device 100 being loaded, the offloading component 120 (e.g., spring 630) compresses and the foot of the user rests on the ground while the offloading device 100 translates a portion of the weight to the lower leg of the user.

The spring compression rod 610 may be made from steel and the spring housing 620 may be made from aluminum. The spring compression rod 610 and spring housing 620 may be made of one or more of steel, aluminum, plastic, fiberglass composite, carbon fiber composite, and/or other metals.

The offloading component 120 may include a knob configured to compress the spring 630 when turned to provide offloading adjustment. The offloading component 120 may include a screw on/off cap that allows for changing the type of spring 630 for offloading adjustment (e.g., replace spring with a spring that has a different spring constant).

The offloading component 120 may include bushings 640 in the spring housing 620 or attached to the spring compression rod 610. The offloading component 120 may include a feature that holds the spring compression rod 610 inside the spring housing 620 when the offloading device 100 is unweighted, such as a cap with a rod-sized hole on the bottom of the spring housing and a bushing 640 secured to the spring compression rod 610 that sits on top of the cap.

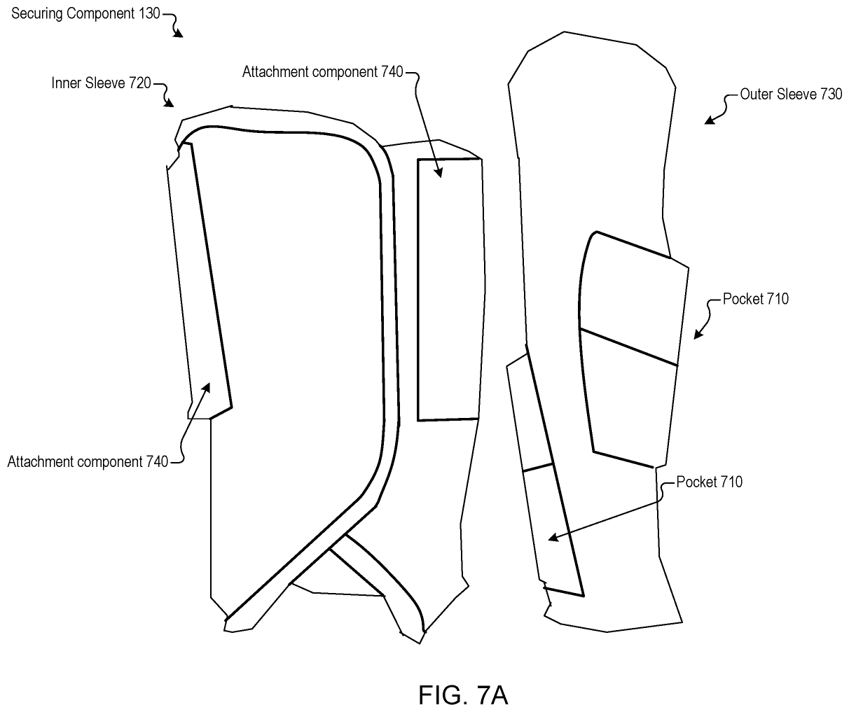

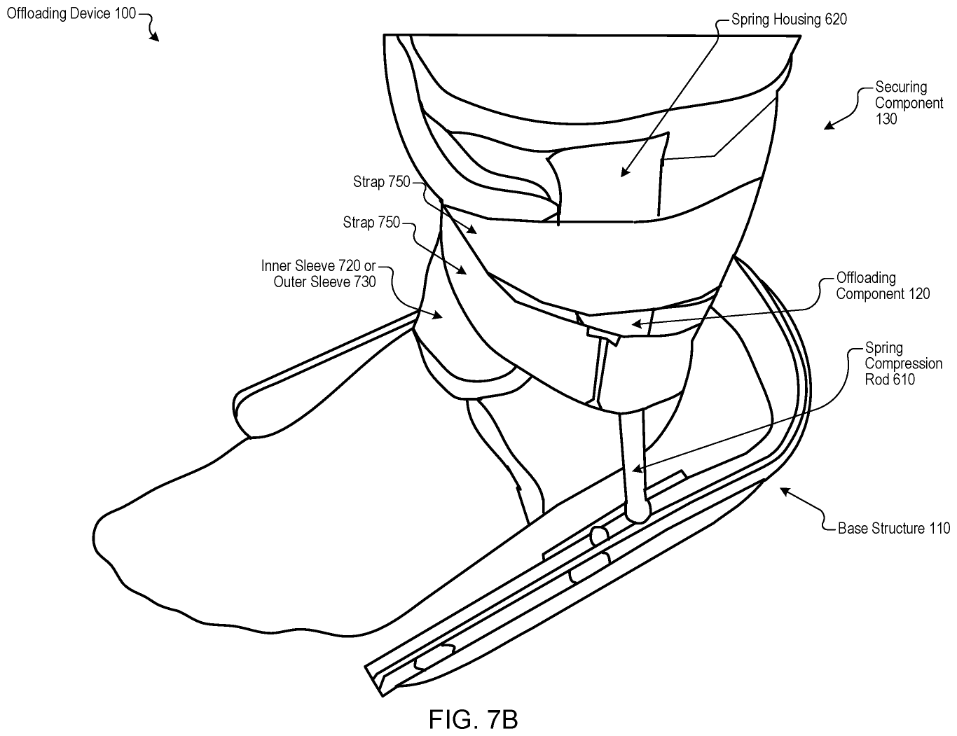

FIGS. 7A-B illustrate securing components 130 (e.g., upper portions) of offloading devices 100, according to certain embodiments. The securing component 130 may include one or more pockets 710 into which a portion (e.g., the spring housing 620) of the offloading component 120 is configured to enter (e.g., slide).

The securing component 130 attaches to the lower leg and to the offloading component 120 (e.g., offloading spring mechanism).

As shown in FIG. 7A, an inner sleeve 720 (e.g., foam material) of the securing component 130 may attach to an outer sleeve 730 (e.g., fabric) of the securing component 130 via attachment component 740 (e.g., via hoop and loop fasteners, via sewing, etc.). The inner sleeve 720 may be coated with a mineral gel that limits slipping on the skin. The offloading device 100 may or may not have the inner sleeve 720.

The outer sleeve 730 in which the pockets 710 are attached can include one or more of fabric, foam, plastic, and/or other materials. The pockets 710 may be attached to the outer sleeve 730 via sewing, stitches, rivets, and/or adhesive.

As shown in FIG. 7B, the securing component 130 may include straps 750 that provide compression that assist in securing the offloading device 100 (e.g., spring housing 620 and/or spring compression rod 610) to the leg of the user. The straps 750 may be sewn to the outer sleeve 730 and/or inner sleeve 720 or may be independent. The number of straps 750 is variable depending on the amount and location of compression to be provided. The compression could be obtained via straps 750, lacing, and/or a pneumatic bladder.

In some embodiments, the offloading component 120 is connected to the securing component 130 via a sleeve, a pivot point, a clamping mechanism, and/or the like.

FIGS. 8A-B illustrate securing components 130 of offloading devices 100, according to certain embodiments. The securing components 130 may include cams 810. The cams 810 may be a first component configured to contact a first side of the leg and a second component configured to contact a second side of the leg, where a connector 820 (e.g., pivoting component) is rotatably connected to the first component, the second component, and the offloading component 120. Responsive to a weight load (e.g., loaded cams 810), the securing component 130 tightens (e.g., cams 810 when loaded) onto the lower leg.

The securing component 130 includes two components (e.g., cams 810) connected by a cam component (e.g., connector 820) that causes compression forces when the offloading device 100 is loaded.

In both FIG. 8A and FIG. 8B, there are two components (e.g., cams 810) that make primary contact with the lower leg. One component (e.g., cam 810) may primarily load on the shin while the other component (e.g., cam 810) may primarily load on the calf. The components (e.g., cams 810) are connected by two cam arms (e.g., connectors 820). The two cam arms (e.g., connectors 820) may pivot about their attachment points on plastic pieces. The offloading component 120 (e.g., offloading spring mechanism) attaches to the cam arms (e.g., connectors 820) with another pivoting joint. The cam arms (e.g., connectors 820) could be one single piece with a bend to allow the single piece to be attached to both sides of the lower leg.

In some embodiments, the securing component 130 includes rigid plastic pieces (e.g., cams 810) to contact the lower leg (e.g., see FIG. 8A). The plastic pieces (e.g., cams 810) may be secured to the lower leg via a compression sleeve that attaches (e.g., via hook and loop fastener) to the plastic pieces (e.g., cams 810). The securing component 130 may or may not have a compression sleeve.

The components of the securing component 130 that contact the lower leg can be manufactured from a variety of materials. For example, FIG. 8A illustrates the use of rigid plastic and FIG. 8B illustrates the use of fabric with a foam insert. Other examples of materials of the securing component include one or more of plastic (e.g., rigid or compliant), foam, fabric, and/or the like.

Although FIGS. 8A-B illustrate a securing component 130 (e.g., brace upper) with multiple components, the securing component 130 could be a soft product in which the cam features are integrated with one or more final components.

The securing component 130 may be of custom manufacture or may have adjustability integrated into the securing component 130 via one or more of straps, multiple sizes, and/or the like. The securing component 130 may be coupled (e.g., rotatably coupled) to an offloading device, such as a spring housing 620 that is coupled to a spring compression rod 610 (e.g., that is coupled to a base structure 110).

FIG. 9 is a block diagram illustrating a computer system 900, according to certain embodiments. In some embodiments, computer system 900 may be connected (e.g., via a network, such as a Local Area Network (LAN), an intranet, an extranet, or the Internet) to other computer systems. In some embodiments, computer system 900 may operate in the capacity of a server or a client computer in a client-server environment, or as a peer computer in a peer-to-peer or distributed network environment. Computer system 900 may be provided by controller 122, a PC, a tablet PC, a Personal Digital Assistant (PDA), a cellular telephone, a web appliance, a server, a network router, switch or bridge, or any device capable of executing a set of instructions (sequential or otherwise) that specify actions to be taken by that device. Further, the term “computer” shall include any collection of computers that individually or jointly execute a set (or multiple sets) of instructions to perform any one or more of the methods described herein.

In a further aspect, the computer system 900 may include a processing device 902, a volatile memory 904 (e.g., random access memory (RAM)), a non-volatile memory 906 (e.g., read-only memory (ROM) or electrically-erasable programmable ROM (EEPROM)), and a data storage device 916, which may communicate with each other via a bus 908.

Processing device 902 may be provided by one or more processors such as a general purpose processor (such as, for example, a complex instruction set computing (CISC) microprocessor, a reduced instruction set computing (RISC) microprocessor, a very long instruction word (VLIW) microprocessor, a microprocessor implementing other types of instruction sets, or a microprocessor implementing a combination of types of instruction sets) or a specialized processor (such as, for example, an application specific integrated circuit (ASIC), a field programmable gate array (FPGA), a digital signal processor (DSP), or a network processor).

Computer system 900 may further include a network interface device 922. Computer system 900 also may include a video display unit 910 (e.g., a liquid crystal display (LCD)), an alphanumeric input device 912 (e.g., a keyboard), a cursor control device 914 (e.g., a mouse), and a signal generation device 920.

In some implementations, data storage device 916 may include a non-transitory computer-readable storage medium 924 on which may store instructions 926 encoding any one or more of the methods or functions described herein, including instructions encoding components of FIG. 1 (e.g., controller 122, etc.) and for implementing methods described herein.

Instructions 926 may also reside, completely or partially, within volatile memory 904 and/or within processing device 902 during execution thereof by computer system 900, hence, volatile memory 904 and processing device 902 may also constitute machine-readable storage media.

While computer-readable storage medium 924 is shown in the illustrative examples as a single medium, the term “computer-readable storage medium” shall include a single medium or multiple media (e.g., a centralized or distributed database, and/or associated caches and servers) that store the one or more sets of executable instructions. The term “computer-readable storage medium” shall also include any tangible medium that is capable of storing or encoding a set of instructions for execution by a computer that cause the computer to perform any one or more of the methods described herein. The term “computer-readable storage medium” shall include, but not be limited to, solid-state memories, optical media, and magnetic media.

FIGS. 10A-C illustrate offloading devices 100, according to certain embodiments. In some embodiments, an offloading device 100 includes a pneumatic air bladder 1010 (e.g., part of the securing component 130). The pneumatic air bladder 1010 may include a bladder configured to wrap around the leg of a user, a tube connected to the bladder, and a pneumatic user interface connected to the tube outside of the boot liner. A user may pump one portion of the pneumatic user interface to increase pressure and may press another portion of the pneumatic user interface to release pressure. In some embodiments, the bladder of the pneumatic air bladder 1010 is configured to be inflated with a bulb that is detachable, where a knob of the pneumatic air bladder 1010 may allow air to go into or out of the bladder.

The methods, components, and features described herein may be implemented by discrete hardware components or may be integrated in the functionality of other hardware components such as ASICS, FPGAs, DSPs or similar devices. In addition, the methods, components, and features may be implemented by firmware modules or functional circuitry within hardware devices. Further, the methods, components, and features may be implemented in any combination of hardware devices and computer program components, or in computer programs.

Unless specifically stated otherwise, terms such as “receiving,” “transmitting,” “generating,” “causing,” “determining,” “performing,” “storing,” “providing,” “displaying,” or the like, refer to actions and processes performed or implemented by computer systems that manipulates and transforms data represented as physical (electronic) quantities within the computer system registers and memories into other data similarly represented as physical quantities within the computer system memories or registers or other such information storage, transmission or display devices. Also, the terms “first,” “second,” “third,” “fourth,” etc. as used herein are meant as labels to distinguish among different elements and may not have an ordinal meaning according to their numerical designation.

Examples described herein also relate to an apparatus for performing the methods described herein. This apparatus may be specially constructed for performing the methods described herein, or it may include a general purpose computer system selectively programmed by a computer program stored in the computer system. Such a computer program may be stored in a computer-readable tangible storage medium.

The methods and illustrative examples described herein are not inherently related to any particular computer or other apparatus. Various general purpose systems may be used in accordance with the teachings described herein, or it may prove convenient to construct more specialized apparatus to perform methods described herein and/or each of their individual functions, routines, subroutines, or operations. Examples of the structure for a variety of these systems are set forth in the description above.

The above description of illustrated implementations of the disclosure, including what is described in the Abstract, is not intended to be exhaustive or to limit the disclosure to the precise forms disclosed. While specific implementations of, and examples for, the disclosure are described herein for illustrative purposes, various equivalent modifications are possible within the scope of the disclosure, as those skilled in the relevant art will recognize.

Various operations are described as multiple discrete operations, in turn, in a manner that is helpful in understanding the present disclosure, however, the order of description should not be construed to imply that these operations are necessarily order dependent. In particular, these operations need not be performed in the order of presentation.

The terms “over,” “under,” “between,” “disposed on,” and “on” as used herein refer to a relative position of one material layer or component with respect to other layers or components. For example, one layer disposed on, over, or under another layer may be directly in contact with the other layer or may have one or more intervening layers. Moreover, one layer disposed between two layers may be directly in contact with the two layers or may have one or more intervening layers. Similarly, unless explicitly stated otherwise, one feature disposed between two features may be in direct contact with the adjacent features or may have one or more intervening layers.

Various embodiments can have different combinations of the structural features described above. For instance, all optional features a device or system described herein can also be implemented in a device or system and specifics in the examples can be used anywhere in one or more embodiments.

While the present disclosure has been described with respect to a limited number of embodiments, those skilled in the art will appreciate numerous modifications and variations therefrom. It is intended that the appended claims cover all such modifications and variations as fall within the true spirit and scope of this present disclosure.

In the description herein, numerous specific details are set forth, such as examples of specific types of material, specific sizes, specific surfaces, specific structures, specific details, specific configurations, specific types, specific system components, specific operations, etc. in order to provide a thorough understanding of the present disclosure. It will be apparent, however, to one skilled in the art that these specific details need not be employed to practice the present disclosure. In other instances, well known components or methods, such as specific and alternative material, sizes, surfaces, structures, details, configurations, types, system components, operations, etc. have not been described in detail in order to avoid unnecessarily obscuring the present disclosure.

Although some of the embodiments herein are described with reference to specific devices or systems, other embodiments are applicable to other types of structures and surfaces. Similar techniques and teachings of embodiments of the present disclosure can be applied to other types of structures and surfaces that can benefit from advantages described herein. In addition, the description herein provides examples, and the accompanying drawings show various examples for the purposes of illustration. However, these examples should not be construed in a limiting sense as they are merely intended to provide examples of embodiments of the present disclosure rather than to provide an exhaustive list of all possible implementations of embodiments of the present disclosure.

As used herein, the terms “substantially,” “about,” and/or the like, in some embodiments refer to a range of 2% greater and 2% less, in some embodiments refer to a range of 5% greater and 5% less, in some embodiments refer to a range of 10% greater and 10% less, in some embodiments refer to a range of 15% greater and 15% less, and in some embodiments refer to a range of 20% greater and 20% less,

Use of the phrase ‘configured to,’ in one embodiment, refers to arranging, putting together, manufacturing, offering to sell, importing and/or designing an apparatus, hardware, logic, or element to perform a designated or determined task. In this example, an apparatus or element thereof that is not operating is still ‘configured to’ perform a designated task if it is designed, coupled, and/or interconnected to perform said designated task.

Furthermore, use of the phrases ‘to,’ ‘capable of/to,’ and or ‘operable to,’ in one embodiment, refers to some apparatus, hardware, and/or element designed in such a way to enable use of the apparatus, hardware, and/or element in a specified manner. Note that use of to, capable to, or operable to, in one embodiment, refers to the latent state of an apparatus, hardware, and/or element, where the apparatus, hardware, and/or element is not operating but is designed in such a manner to enable use of an apparatus in a specified manner.

Reference throughout this specification to “one embodiment,” “an embodiment,” or “some embodiments” means that a particular feature, structure, or characteristic described in connection with the embodiment is included in at least one embodiment of the present disclosure. Thus, the appearances of the phrases “in one embodiment,” “in an embodiment,” and “in some embodiments” in various places throughout this specification are not necessarily all referring to the same embodiment. Furthermore, the particular features, structures, or characteristics can be combined in any suitable manner in one or more embodiments.

In the foregoing specification, a detailed description has been given with reference to specific exemplary embodiments. It will, however, be evident that various modifications and changes can be made thereto without departing from the broader spirit and scope of the disclosure as set forth in the appended claims. The specification and drawings are, accordingly, to be regarded in an illustrative sense rather than a restrictive sense. Furthermore, the foregoing use of embodiment and other exemplarily language does not necessarily refer to the same embodiment or the same example, but can refer to different and distinct embodiments, as well as potentially the same embodiment.

The words “example” or “exemplary” are used herein to mean serving as an example, instance or illustration. Any aspect or design described herein as “example’ or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an implementation” or “one implementation” throughout is not intended to mean the same embodiment or implementation unless described as such. Also, the terms “first,” “second,” “third,” “fourth,” etc. as used herein are meant as labels to distinguish among different elements and can not necessarily have an ordinal meaning according to their numerical designation.

What is claimed is:

1. An offloading device comprising:
a base structure configured to be disposed at least partially around a foot of a user;
a securing component configured to secure around a leg of the user; and
one or more offloading components coupled between the base structure and the securing component to offload weight from the foot of the user.

2. The offloading device of claim 1, wherein the base structure is further configured to be disposed under the foot of the user, wherein the offloading device is configured to maintain the foot above the base structure responsive to the one or more offloading components being in an unloaded state, and wherein the offloading device is an offloading boot.

3. The offloading device of claim 1, wherein the base structure is configured to be disposed at least partially around the foot of the user without being disposed under the foot of the user, wherein the offloading device is configured to maintain the foot above a surface on which the base structure is disposed responsive to the one or more offloading components being in an unloaded state, and wherein the offloading device is an offloading brace.

4. The offloading device of claim 1, wherein the one or more offloading components comprises:
a first offloading component comprising one or more first springs, the first offloading component being disposed on a first side of the offloading device; and
a second offloading component comprising one or more second springs, the second offloading component being disposed on a second side of the offloading device, the one or more first springs and the one or more second springs being configured to offload the weight from the foot of the user.

5. The offloading device of claim 1, wherein the securing component comprises one or more of hook and loop fasteners, laces, tightening buckle, ratcheting system, or a pneumatic bladder, and wherein the securing component is configured to secure around the leg of the user to maintain the foot elevated above one or more of the base structure or a surface on which the base structure is disposed responsive to the one or more offloading components being in an unloaded state.

6. The offloading device of claim 1, wherein the securing component comprises a first cam configured to be disposed on a front portion of the leg and a second cam configured to be disposed on a back portion of the leg, wherein the first cam and the second cam comprise at least one of rigid components or fabric components, wherein the first cam and the second cam are connected by a first connector and a second connector, wherein a first offloading component of the one or more offloading components is coupled to the first connector, and wherein a second offloading component of the one or more offloading components is coupled to the second connector.

7. The offloading device of claim 1, wherein the securing component comprises one or more fasteners and a compressible material, wherein the compressible material is configured to be disposed between the leg and the one or more fasteners.

8. The offloading device of claim 1, wherein the one or more offloading components comprise one or more of a motorized piston or a pneumatic device.

9. The offloading device of claim 1, wherein the offloading component is adjustable to adjust a quantity of the weight to be offloaded from the foot of the user, wherein the one or more offloading components comprises one or more of:
a motor that adjusts spring load;
a piston that adjusts spring load;
an electrical actuator that adjusts compression of the offloading component;
a knob that is configured to compress one or more first springs of the one or more offloading components;
a ratcheting system that adjusts spring tension or compression of the one or more offloading components;
the one or more first springs that are replaceable with one or more second springs that have different spring rates than the one or more first springs; or
an adjustment screw.

10. The offloading device of claim 1, wherein the offloading component comprises one or more of a controller, an electronic valve of a pneumatic bladder, a pressure sensor, a heart rate sensor, or a distance sensor.

11. The offloading device of claim 1, wherein the base structure comprises a rockered base constructed of plastic with a rubber outsole and a foam foot bed.

12. The offloading device of claim 1, wherein a first offloading component of the one or more offloading components comprises:
an upper bracket coupled to the securing component;
a lower bracket coupled to the base structure;
an adjustment screw secured to the lower bracket and routed through the upper bracket; and
one or more springs disposed between the upper bracket and the lower bracket to offload weight from the foot of the user.

13. The offloading device of claim 12, wherein the first offloading component further comprises one or more guide pins disposed between the upper bracket and the lower bracket, wherein each of the one or more springs is disposed around a corresponding guide pin of the one or more guide pins.

14. The offloading device of claim 1, wherein the offloading component comprises a controller configured to receive one or more of user input or sensor data and to cause, based on the one or more of user input or sensor data, adjustment of a quantity of the weight to be offloaded from the foot of the user.

15. An offloading device comprising:
a base structure configured to be disposed on a surface;
a securing component configured to secure to a leg of a user to maintain a foot of the user elevated above the surface responsive to the offloading device being in an unloaded state, and wherein the foot of the user is to be disposed on the base structure or on the surface responsive to the offloading device being in a loaded state.

16. The offloading device of claim 15 further comprising:
a first offloading component connected to the base structure and the securing component on a first side of the leg; and
a second offloading component connected to the base structure and the securing component on a second side of the leg.

17. The offloading device of claim 16, wherein one or more corresponding components of the first offloading component and the second offloading component are to compress responsive to the offloading device being in the loaded state.

18. An offloading device comprising:
a base structure configured to be disposed at least partially around a foot of a user;
a plurality of offloading components coupled to the base structure and coupled to a leg of the user to maintain the foot in an elevated state responsive to the offloading device being in an unloaded state, wherein the plurality of offloading components are configured to offload weight from the foot of the user.

19. The offloading device of claim 18, wherein the plurality of offloading components comprise a first offloading component and a second offloading component, wherein the first offloading component comprises a first spring and the second offloading component comprises a second spring, wherein the first and second spring are to compress responsive to the offloading device being in a loaded state to offload the weight from the foot of the user.

20. The offloading device of claim 18, wherein the plurality of offloading components are coupled to the leg of the user via one or more of fasteners or cams.
